(19) United States
(12) Patent Application Publication
(10) Pub. No.: US 2005/0064429 A1
(43) Pub. Date: Mar. 24, 2005

(54) Method for diagnosing and treating predisposition for accelerated autosomal dominant polycystic kidney disease

(76) Inventors: Olivier Devuyst, Bruxelles (BE); Alexandre Persu, Zaventem (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/506,359

(22) PCT Filed: Mar. 7, 2002
(86) PCT No.: PCT/EP02/02505

(51) Int. Cl. C12Q 1/68; C07H 21/04

(52) U.S. Cl. 435/6; 536/23.1

(57) ABSTRACT

A method for diagnosing a predisposition for accelerated autosomal dominant polycystic kidney disease (ADPKD) in a human male subject by detecting the Glu 298 Asp polymorphism of the ENOS gene is described. A diagnostic kit for detecting predisposition for accelerated ADPKD in a human subject is also disclosed. In addition, a method for treating a human subject predisposed to develop accelerated APDKD using NO-enhancing compounds is provided.

### FIELD OF THE INVENTION

Present invention relates in one aspect to a method for diagnosing a predisposition for accelerated autosomal dominant polycystic kidney disease (ADPKD) in a human subject. In another aspect, present invention relates to a diagnostic kit for detecting predisposition for accelerated ADPKD in a human subject. In another aspect, present invention provides a method for treating a human subject predisposed to develop accelerated APDKD using NO-enhancing compounds.

### BACKGROUND OF THE INVENTION

Autosomal dominant polycystic kidney disease (ADPKD) is one of the most common monogenic hereditary diseases (prevalence: 1:500-1:1000). Its principal clinical manifestation is the development of multiple cysts in both kidneys. These cysts grow slowly over decades, until renal failure occurs. ADPKD-associated renal cysts may enlarge to contain several liters of fluid and the kidneys usually enlarge progressively causing pain. Other abnormalities such as pain, hematuria, renal and urinary infection, renal tumors, salt and water imbalance and hypertension frequently result from the renal defect. By the age of 60 years, about half the patients with ADPKD have progressed to end-stage renal disease (ESRD). “End-stage renal disease” (ESRD) is defined as the condition when life becomes impossible without replacement of renal functions either by kidney dialysis or kidney transplantation. In Europe and North-America, ADPKD is responsible for 10% of the patients requiring renal replacement therapy (Pirson et al. 1998, Oxford Textbook of Clinical Nephrology. Oxford University Press, Oxford, UK, pp. 2393-2415). In Europe, more particularly in Belgium and France, 10.000 and 50.000 patients, respectively, are afflicted by ADPKD.

To date, genetic and molecular studies have revealed that most forms of ADPKD are associated with mutations in two genes, the polycystic kidney disease 1 (PKD1) and the polycystic kidney disease 2 (PKD2) genes. Mutations in the PKD1 gene are suspected of causing 80-90% of all cases of ADPKD. Mutations in the PKD2 gene account for the vast majority of the remaining cases (Pirson et al. 1998, Oxford Textbook of Clinical Nephrology. Oxford University Press, Oxford, UK, pp. 2393-2415; Kimberling et al. 1993, Genomics 18:467, Peters and Sandkuijl 1992, Contrib. Nephrol. 97:128).

One of the most striking features in ADPKD is the substantial variability in the severity of renal phenotype, primarily assessed by the age at ESRD. This variability is observed among families, family members, and even dizygotic twins (Milutinovic et al. 1992, Am. J. Kidney Dis. 19:465-472; Torra et al. 1995, Contrib. Nephrol. 115:97-101). Interfamilial phenotypic variability may be explained by distinct effects of mutations in PKD1 and PKD2 and also by the nature of the mutation itself. Indeed, PKD2 is clinically milder than PKD1 disease, as witnessed by a later age at ESRD and a lower prevalence of hypertension (Hateboer et al. 1999, Kidney Int. 5634-40). In addition, different mutations in the same gene are associated with differences in the mean age at ESRD in both PKD1 (Hateboer et al. 1999, Kidney Int. 56:34-40) and PKD2families (Hateboer et al. 1999, Lancet 353103-107).

Compositions and methods for the diagnosis and treatment of ADPKD, associated with mutations in the PDK1 and/or the PDK2 gene, are known, as for instance described in U.S. Pat. No. 6,228,591, U.S. Pat. No. 6,031,088 and WO-A2-9534649. Furthermore, document WO-A1-9534573 describes high throughput assays to identify compounds that interfere with PKD activity and inhibit the expression, synthesis and/or bioactivity of the PKD gene product. These compounds could be used therapeutically to treat subjects afflicted by polycystic kidney disease.

However, variability in the severity of renal phenotype, primarily assessed by the age at ESRD, can not be completely attributed to PKD1 or PKD2-associated mutations. Intrafamilial phenotypic variability could be explained by at least two mechanisms: the second hit event, and the effect of modifier genes. If cyst formation is triggered by a second hit, i.e. a somatic mutation in the allele unaffected by germline mutation (Qian, et al. 1996, Cell 87:979-987), micro-environmental or genetic factors determining the rate of second hit could be a valuable explanation. Alternatively, modifier genes could exert either a protective or a deleterious effect on the renal phenotype. The role of modifier genes has been shown in other hereditary diseases, including cystic fibrosis, familial mediterranean fever or familial hypercholesterolemia (Nadeau 2001, Nat. Rev. Genet. 2:165-174), and modifier loci have recently been demonstrated in several mouse models of polycystic kidney disease (Woo et al. 1997, J. Clin. Invest. 100:1934-1940; Upadhya et al. 1999, Genomics 58:129-137).

By far the most common source of variation in the genome are single nucleotide polymorphisms of SNPs. In general single nucleotide polymorphism (SNP) account for approximately 90% of human DNA polymorphisms (Collins et al. 1998, Genome Res. 8:1229-1231). SNPs are single base pair positions in genomic DNA at which different sequence alternatives (alleles) exist in a population. As used herein, the term “single nucleotide polymorphism” or “SNP” includes single nucleotide substitutions (e.g. A->G). Nucleotide substitutions are of two types. A transition is the replacement of one purine by another purine or one pyrimidine by another pyrimidine. A transversion is the replacement of a purine for a pyrimidine or vice versa. Numerous methods exist for the detection of SNPs within a nucleotide sequence. A review of many of these methods can be found in Landegren et al. 1998, Genome Res. 8:769-776).

Present invention is based on the observation that a particular gene, the ENOS gene, which encodes an endothelial nitric oxide synthase (eNOS) has a modifier and deleterious effect in ADPKD. More particularly, the inventor discovered that a single nucleotide polymorphism in this gene influences the progression of ADPKD. Therefore, human subjects carrying a single nucleotide polymorphism of this ENOS gene may be predisposed to develop the ADPKD disease much faster compared to subject carrying the wild-type ENOS gene. “Accelerated ADPKD” as defined herein is the faster development of the disease whereby afflicted patients have a considerable 5 to 10 years earlier need for dialysis. Thus, a faster development of ADPKD is characterized by faster renal decline and a 5 to 10 year lower mean age of the ADPKD-afflicted subjects at end stage renal disease. Besides serious medical and health implications for afflicted patients, the accelerated development of the disease also involves important financial and socio-economic consequences, as dialysis treatments are extremely expensive.

The ENOS gene encodes an endothelial nitric oxide synthase (eNOS), a Ca²⁺-dependent enzyme which catalyses the production of nitric oxide (NO) in endothelial cells. The release of nitric oxide (NO) by endothelial cells plays a critical role in the control of local haemodynamics and systemic blood pressure (Vallance et al. 1989, Lancet2:997-1000; Forte et al. 1997, Lancet 349:837-842). NO possesses vasodilatory and antiantherogenic properties. Therefore, an altered eNOS function may accelerate associated with endothelial dysfunction. In general nitric oxide synthase genes have been associated with hypertension, renal failure and cardiovascular diseases (Soubrier 1998, Hypertension 31:189-193).

Many polymorphisms have been reported in the sequence of the ENOS gene. Furthermore, document WO-A2-0153537 discloses methods and kits for detecting the presence or absence of SNPs of the ENOS gene, which are associated with a predisposition to several diseases including hypertension, end stage renal disease due to hypertension, non-insulin dependent diabetes mellitus, end stage renal disease due to non-insulin dependent diabetes mellitus, breast cancer, lung cancer or prostate cancer. This document also discloses methods for the treatment and/or prophylaxis of above-mentioned diseases, conditions, or disorders associated with the single nucleotide polymorphisms.

However, the document WO-A-0153537 has not revealed or described a possible association of a SNP of the ENOS gene with ADPKD, as it does not even mention ADPKD at all, which is a the most common hereditary renal disease with a important prevalence of 1:500 to 1:1,000. Moreover, the document covers all SNPs of the ENOS gene in general and not a single SNP in particular and it does not demonstrate or even suggests the pathophysiological role of a SNP and its translation to a given treatment in ADPKD. In fact, up to date none of the known ENOS polymorphisms was described to influence the progression of ADPKD or to be associated with accelerated ADPKD and no treatment at all is known to slow down ADPKD progression. Therefore, there is a need and potential benefit of genotyping ENOS in order to find association of SNPs of the ENOS gene with ADPKD and disease progression.

It is an objective of the present invention to detect polymorphisms of the ENOS gene which are associated with a predisposition for accelerated development of ADPKD. More particularly, it is an objective of the present invention to provide means and methods to rapidly detect ADPKD-patients predisposed to develop an accelerated ADPKD disease, so that these subjects can be provided with a suitable treatment as soon as the condition of APPKD can be diagnosed. One of the main interests of the diagnostic kit and diagnostic method is to detect the predisposition to accelerated ADPKD early enough, in order to be able to start a treatment which may slow down the progression of the disease as soon as possible.

Another object of the present invention is to provide a method for treating predisposition to accelerated ADPKD in human subject. This treatment could result in a delay of the progression of the disease. An ideal approach for preventing accelerated ADPKD would be the identification of the presence of a polymorphism that predisposes an individual for accelerated ADPKD early enough to be able to counteract this predisposition. Knowledge of accelerated ADPKD predisposing polymorphisms is essential for truly effective delay, or, ideally, prevention of accelerated ADPKD.

### SUMMARY OF THE INVENTION

The present inventor has discovered that a common SNP in exon 7 of the ENOS gene has a strong risk factor for accelerating ADPKD. This polymorphism results in the glutamate to aspartate amino acid substitution at position 298 in the amino acid sequence. More particularly, the present inventor discovered that this Glu 298 Asp polymorphism is associated with a 5 to 10-year lower mean age at ESRD of a subject afflicted with ADPKD. This Glu 298 Asp polymorphism of the ENOS gene is known, and has been associated with other conditions characterized by endothelial dysfunction, including hypertension (Uwabo et al. 1998, Am. J. Hypertens. 11:125-128; Miyamoto et al. 1998, Hypertension 32:3-8), myocardial infarction (Hibi et al. 1998, Hypertension 32:521-526; Shimasaki et al. 1998, J. Am. 0 Coll. Cardiol. 31:1506-151; Hingorani et al. 1999, Circulation 100:1515-1520; Wang et al. 1996, Nat. Med. 2:41-45), carotid atheriosclerosis (Miyamoto et al. 2000, Hum. Mol. Genet. 9:2629-2637), coronary spasm (Yoshimura et al. 1998, Hum. Genet. 103:65-69), preeclampsia, enhanced vasoconstriction by phenylephrine (Philip et al. 1999, Circulation 99:3096-3098), and brain infarction (Elbaz et al. 2000, Stroke 31:1634-1639). However, so far this Glu 298 Asp polymorphism had not yet been associated with ADPKD.

Based upon the knowledge of this novel ADPKD accelerating factor, a diagnostic method and kit were designed for the specific detection and genotyping of the ENOS gene allele status of humans afflicted by ADPKD. The determination of this ENOS gene allele status with such tests is useful in the determination of the individual predisposition to accelerated ADPKD and useful for the optimization of therapies.

Accordingly, in a first aspect, present invention relates to a method for diagnosing a predisposition for accelerated autosomal dominant polycystic kidney disease in a human subject comprising
obtaining a biological sample containing nucleic acid from said subject, and
detecting in said nucleic acid the presence of a single nucleotide polymorphism in the ENOS gene sequence, or the complement thereof.
   Said single nucleotide polymorphism is associated with a predisposition of said disease.

In a second aspect, present invention relates to a diagnostic kit for carrying out the method of the invention that comprises
one isolated polynucleotide of at least 10 contiguous nucleotides of the ENOS gene sequence or the complement thereof, and containing at least one single nucleotide polymorphism, wherein said single nucleotide polymorphism is associated with a predisposition for accelerated ADPKD,
suitable reagents, and
instructions for using said polynucleotide for detecting the presence of said single nucleotide polymorphism in a biological sample containing said nucleic acid.

This kit allows rapid, reliable and easy screening of a large population of human subjects on the presence of said polymorphism of the ENOS gene.

In a third aspect present invention relates to a method for treatment of a human subject predisposed to develop accelerated autosomal dominant polycystic kidney disease comprising
determining the predisposition of said subject by carrying out the method of the present invention, and
administrating at least one NO-enhancing compound in said subject in need of said treatment.
   This method of treatment allows counter acting the effect of the SNP of the ENOS gene associated with the accelerated ADPKD as soon as the condition of APPKD is diagnosed, and provides the possibility to postpone ADPKD progression in afflicted human subjects. More particularly, the increase in NO levels in a patient in need of such a treatment may comprise the administration of a substrate for eNOS or NO donors.

Further scope of the applicability of the present invention will become apparent from the detailed description provided below. It should be understood, however, that the following detailed description and examples, while indicating preferred embodiments of the invention, are given by way of illustration only, since various changes and modifications within the spirit and scope of the invention will become apparent to those skilled in the art from the following detailed description.

### DETAILED DESCRIPTION OF THE INVENTION

The finding that a polymorphic variation in the ENOS gene in ADPKD-afflicted human individuals is a ADPKD accelerating factor provides a very potent tool for improving the diagnostic and preventive therapy of accelerated ADPKD. The diagnosis of the polymorphism permits a more focussed therapy of APDKD-patients harboring the polymorphism, e.g. by opening the possibility to apply individual dose regiments of drugs as soon as possible after ADPKD is diagnosed. Furthermore diagnostic tests to genotype the ENOS gene polymorphism may help to correlate the genotypes with drug activity or side effects.

In a first embodiment, present invention relates to a method for diagnosing a predisposition for accelerated ADPKD in human subject comprising
obtaining a biological sample containing nucleic acid from said subject, and
detecting in said nucleic acid the presence of a single nucleotide polymorphism in the ENOS gene sequence, or the complement thereof.

As used herein, the term “predisposition” refers to the likelihood that an individual subject will develop a particular disease. In the context of present invention the term “accelerated ADPKD” is defined as a faster renal decline and a 5 to 10-year lower mean age at ESRD of a human subject afflicted by ADPKD. “Sequence” means the linear order in which monomers occur in a polymer, for example, the order of amino acids in a polypeptide or the order of nucleotides in a polynucleotide. “Polymorphism” refers to a set of genetic variants at a particular genetic locus among individuals in a population. As used herein, “single nucleotide polymorphism” or “SNP” includes single base pair substitutions in genomic DNA. The nucleotide and amino acid sequences of the human ENOS gene are known and described by Miyahara et al. 1994 (Genbank Acc. No D26607).

In a preferred embodiment, the present invention provides a method wherein the nucleic acid in the biological sample is DNA, cDNA, RNA or mRNA. For the assay of genomic DNA, virtually any biological sample containing genomic DNA (e.g. not pure red blood cells) can be used. For example, and without limitation, genomic DNA can be conveniently obtained from whole blood, semen, saliva, tears, urine, fecal material, sweat, buccal cells, skin or hair. For assays using cDNA or mRNA, the target nucleic acid can be obtained from cells or tissues that express the target sequence. One preferred source and quantity of DNA is 10 to 30 ml of anti-coagulated whole blood, since enough DNA can be extracted from leukocytes in such a sample to perform many repetitions of the analysis contemplated herein.

In a more preferred embodiment, the present invention is related to a method wherein said single nucleotide polymorphism corresponds to the Glu 298 Asp polymorphism of the ENOS gene. This method of diagnosis provides the possibility to detect human subjects carrying the Glu 298 Asp polymorphism at an early stage and thus to early detect a predisposition for accelerated ADPKD.

There is growing recognition that SNPs can provide a powerful tool for the detection of individuals whose genetic make-up alters their susceptibility to certain diseases. In accordance with the association of SNPs with diseases, present invention provides a link between the Glu 298 Asp polymorphism of the ENOS gene and a progression of ADPKD. ADPKD is associated with conditions characterized by endothelial dysfunction, such as an alteration of the endothelium-dependent vasodilation. This condition has been attributed to a decreased production of NO by ENOS during ADPKD (Wang et al. 2000, J Am Soc Nephrol. 11:1371-1376). Thus, small changes in NO levels, may play a role in the progression of renal disease, and ENOS may be a modifier gene in ADPKD. Also in accordance with this SNP-disease association, present invention further provides the observation that Ca²⁺-dependent NOS activity decreases in renal artery samples from ADPKD patients harboring the Asp 298 allele, in association with post-translational modifications and a partial cleavage of eNOS. Therefore, the resulting decrease in NO production may enhance the endothelial dysfunction associated with ADPKD, leading to alteration of intrarenal and/or systemic haemodynamics. This in turn may result in a faster decline in renal function.

Several methods have been developed to detect unknown as well as known SNPs (reviewed by Landegren et al. 1998, Genome Res. 8:769-776). Determination of unknown genetic variants and in particular SNPs, whitin a particular nucleotide sequence among a population may be determined by any method known in the art, for example and without limitation: direct sequencing, restriction fragment length polymorphism (RFLP), denaturing gradient gel electrophoresis (DGGE), heteroduplex analysis (HET), chemical cleavage analysis (CCM) and ribonuclease cleavage.

Methods for direct sequencing of nucleotide sequences are well known to those skilled in the art and can be found for example in Ausubel et al. 1995 (eds., Short Protocols in Molecular Biology, 3rd ed., Wiley) and Sambrook et al. 1989 (Molecular Cloning, 2'd ed., Chap. 13, Cold Spring Harbor Laboratory Press). Sequencing can be carried out by any suitable method, for example, dideoxy sequencing (Sanger et al. 1977, Proc. Natl. Acad. Sci. USA 74:5463-5467), chemical sequencing (Maxam and Gilbert 1977, Proc. Natl. Acad. Sci. USA 74:560-564) or variations thereof.

SNPs can be for instance detected by restriction fragment length polymorphism (RFLP) (U.S. Pat. Nos. 51,324,631 and 5,645,995). RFLP analysis of the SNPs, however, is limited to cases where the SNP either creates or destroys a restriction enzyme cleavage site. RFLP analysis is useful for detecting the presence of genetic variants at a locus in a population when the variants differ in the size of a probed restriction fragment within the locus, such that the difference between the variants can be visualized by electrophoresis. Such differences will occur when a variant creates or eliminates a restriction site within the probed fragment. RFLP analysis is also useful for detecting a large insertion or deletion within the probed fragment.

Denaturing gradient gel electrophoresis (DGGE) can detect single base mutations based on differences in migration between homo- and heteroduplexes (Myers et al. 1985, Nature 313:495-498). The DNA sample to be tested is hybridized to a labeled wild type probe. The duplexes formed are then subjected to electrophoresis through a polyacrylamide gel that contains a gradient of DNA denaturant parallel to the direction of electrophoresis. Heteroduplexes formed due to single base variations are detected on the basis of differences in migration between the heteroduplexes and the homoduplexes formed.

In heteroduplex analysis (HET) (Keen et al. 1991, Trends Genet. 7:5), genomic DNA is amplified by the polymerase chain reaction followed by an additional denaturing step which increases the chance of heteroduplex formation in heterozygous individuals. The PCR products are then separated on Hydrolink gels where the presence of the heteroduplex is observed as an additional band.

Chemical cleavage analysis (CCM) is based on the chemical reactivity of thymine M when mismatched with cytosine, guanine or thymine and the chemical reactivity of cytosine(C) when mismatched with thymine, adenine or cytosine (Cotton et al. 1988, Proc. Natl. Acad. Sci. USA 85:4397-4401). Duplex DNA formed by hybridization of a wild type probe with the DNA to be examined, is treated with osmium tetroxide for T and C mismatches and hydroxylamine for C mismatches. T and C mismatched bases that have reacted with the hydroxylamine or osmiumtetroxide are then cleaved with piperidine. The cleavage products are then analyzed by gelelectrophoresis.

Ribonuclease cleavage involves enzymatic cleavage of RNA at a single base mismatch in an RNA:DNA hybrid (Myers et al. 1985, Science 230:1242-1246). A 32P-labeled RNA probe complementary to the wild type DNA is annealed to the test DNA and then treated with ribonuclease A. If a mismatch occurs, ribonuclease A will cleave the RNA probe and the location of the mismatch can then be determined by size analysis of the cleavage products following gel electrophoresis.

In addition to the already discussed methods for the detection of polymorphisms, several methods have been developed to detect known SNPs. Many of these assays have been reviewed by Landegren et al. 1988 (Genome research 8:759-776), and will only be briefly reviewed here.

Numerous assays based on hybridization have also been developed to detect SNPs. Hybridization assays may for an example comprise the multiplexed allele-specific diagnostic assay (MASDA) (U.S. Pat. No. 5,834,181; Shuber et al. 1997, Hum. Molec. Genet. 6:337-347). In MASDA, samples from multiplex PCR are immobilized on a solid support. A single hybridization is conducted with a pool of labeled allele specific oligonucleotides (ASO). Any ASO that hybridizes to the samples are removed from the pool of ASOs. The support is then washed to remove unhybridized ASOs remaining in the pool. Labeled ASOs remaining on the support are detected and eluted from the support. The eluted ASOs are then sequenced to determine the mutation present.

Two assays depend on hybridization-based allele-discrimination during PCR. The TaqMan assay (U.S. Pat. No. 5,962,233; Livak et al. 1995, Nature Genet. 9:341-342) uses allele specific (ASO) probes with a donor dye on one end and an acceptor dye on the other end such that the dye pair interact via fluorescence resonance energy transfer (FRET). A target sequence is amplified by PCR modified to include the addition of the labeled ASO probe. The PCR conditions are adjusted so that a single nucleotide difference will effect binding of the probe. Due to the 5′ nuclease activity of the Taq polymerase enzyme, a perfectly complementary probe is cleaved during PCR while a probe with a single mismatched base is not cleaved. Cleavage of the probe dissociates the donor dye from the quenching acceptor dye, greatly increasing the donor fluorescence.

An alternative to the TaqMan assay is the molecular beacons assay (U.S. Pat. No. 5,925,517; Tyagi et al. 1998, Nature Biotech. 16:49-53). In the molecular beacons assay, the ASO probes contain complementary sequences flanking the target specific species so that a hairpin structure is formed. The loop of the hairpin is complimentary to the target sequence while each arm of the hairpin contains either donor or acceptor dyes. When not hybridized to a donor sequence, the hairpin structure brings the donor and acceptor dye close together thereby extinguishing the donor fluorescence. When hybridized to the specific target sequence, however, the donor and acceptor dyes are separated with an increase in fluorescence of up to 900 fold. Molecular beacons can be used in conjunction with amplification of the target sequence by PCR and provide a method for real time detection of the presence of target sequences or can be used after amplification.

High throughput screening for SNPs that affect restriction sites can also be achieved by Microtiter Array Diagonal Gel Electrophoresis (MADGE) (Day and Humphries 1994, Anal. Biochem., 222:389-395). In this assay, restriction fragment digested PCR products are loaded onto stackable horizontal gels with the wells arrayed in a microtiter format. During electrophoresis, the electric field is applied at an angle relative to the columns and rows of the wells allowing products from a large number of reactions to be resolved.

In addition, mismatch distinction by polymerases and ligases have also been used to detect SNPs. The polymerization step in PCR places high stringency requirements on correct base pairing of the 3′ end of the hybridizing primers. This has allowed the use of PCR for the rapid detection of single base changes in DNA by using specifically designed oligonucleotides in a method variously called PCR amplification of specific alleles (PASA) (Sommer et al. 1989, Mayo Clin. Proc. 64:1361-1372; Sarker et al. 1990, Anal. Biochem), allele-specific amplification (ASA), allele-specific PCR, and amplification refractory mutation system (ARMS) (Newton et al. 1989, Nuc. Acids Res; Nichols et al. 1989, Genomics; Wu et al. 1989 Proc. Natl. Acad. Sci. USA). In these methods, an oligonucleotide primer is designed that perfectly matches one allele but mismatches the other allele at or near the 3′ end. This results in the preferential amplification of one allele over the other. By using three primers that produce two different lysized products, it can be determine whether an individual is homozygous or heterozygous for the mutation (Dutton and Sommer 1991, DioTechniques,11:700-702). In another method, termed bi-PASA, four primers are used; two outer primers that bind at different distances from the site of the SNP and two allele specific inner primers (Liu et al. 1997, Genome Res. 7:389-398). Each of the inner primers have a non-complementary 5′ end and form a mismatch near the 3′ end if the proper allele is not present. Using this system, zygosity is determined based on the size and number of PCR products produced.

The joining by DNA ligases of two oligonucleotides hybridized to a target DNA sequence is quite sensitive to mismatches close to the ligation site, especially at the 3′ end. This sensitivity has been utilized in the oligonucleotide ligation assay (OLA)(Landegren et al. 1988, Science 241:1077-1080) and the ligase chain reaction (LCR; Barany 1991, Proc. Natl. Acad. Sci. USA, 88:189-193). In OLA, the sequence surrounding the SNP is first amplified by PCR, whereas in LCR, genomic DNA can by used as a template.

In one method for mass screening for SNPs based on the OLA, amplified DNA templates are analyzed for their ability to serve as templates for ligation reactions between labeled oligonucleotide probes (Samotiaki et al. 1994, Genomics 20:238-242). In this assay, two allele-specific probes labeled with either of two lanthanide labels (europium or terbium) compete for ligation to a third biotin labeled phosphorylated oligonucleotide and the signals from the allele specific oligonucleotides are compared by time-resolved fluorescence. After ligation, the oligonucleotides are collected on an avidin-coated 96-pin capture manifold. The collected oligonucleotides are then transferred to microtiter wells in which the europium and terbium ions are released. The fluorescence from the europium ions is determined for each well, followed by measurement of the terbium fluorescence.

In alternative gel-based OLA assays, numerous SNPs can be detected simultaneously using multiplex PCR and multiplex ligation (U.S. Pat. No. 5,830,711; Day et al. 1995, Genomics 29:152-162; Grossman et al. 1994, Nuc. Acids Res. 22:4527-534). In these assays, allele specific oligonucleotides with different markers, for example, fluorescent dyes, are used. The ligation products are then analyzed together, for example, by electrophoresis on an automatic DNA sequencer distinguishing markers by size and alleles by fluorescence. In the assay by Grossman et al. (1994) mobility is further modified by the presence of a non-nucleotide mobility modifier on one of the oligonucleotides.

A further modification of the ligation assay has been termed the dye-labeled oligonucleotide ligation (DOL) assay (U.S. Pat. No. 5,945,283; Chen et al. 1998; Genome Res. 8:549-556). DOL combines PCR and the oligonucleotide ligation reaction in a two-stage thermal cycling sequence with fluorescence resonance energy transfer (FRET) detection. In the assay, labeled ligation oligonucleotides are designed to have annealing temperatures lower than those of the amplification primers. After amplification, the temperature is lowered to a temperature where the ligation oligonucleotides can anneal and be ligated together. This assay requires the use of a thermostable ligase and a thermostable DNA polymerase without 5′ nuclease activity. Because FRET occurs only when the donor and acceptor dyes are in close proximity, ligation is inferred by the change in fluorescence.

In another method for the detection of SNPs termed mini sequencing, the target-dependent addition by a polymerase of a specific nucleotide immediately downstream (3′) to a single primer is used to determine which allele is present (U.S. Pat. No. 5,846,710). Using this method, several SNPs can be analyzed in parallel by separating locus specific primers on the basis of size via electrophoresis and determining allele specific incorporation using labeled nucleotides.

Determination of individual SNPs using solid phase mini sequencing has been described by Syvanen et al. 1993 (Am. J. Hum. Genet. 52:46-59). In this method, the sequence including the polymorphic site is amplified by PCR using one amplification primer, which is biotinylated on its 5′ end. The biotinylated PCR products are captured in streptavidin-coated microtitration wells, the wells washed, and the captured PCR products denatured. A sequencing primer is then added whose 3′ end binds immediately prior to the polymorphic site, and the primer is elongated by a DNA polymerase with one single labeled dNTP complementary to the nucleotide at the polymorphic site. After the elongation reaction,the sequencing primer is released and the presence of the labeled nucleotide detected. Alternatively, dye labeled dideoxynucleoside triphosphates (ddNTPs) can be used in the elongation reaction (U.S. Pat. No. 5,888,819; Shumaker et al. 1996, Human Mut. 7:346-354). In this method, incorporation of the ddNTP is determined using an automatic gel sequencer.

Mini sequencing has also been adapted for use with microarrays (Shumaker et al. 1996, Human Mut. 7:346-354). In this case, elongation (extension) primers are attached to a solid support such as a glass slide. Methods for construction of oligonucleotide arrays are well known to those of ordinary skill in the art and can be found, for example, in Nature Genetics, Suppl., Vol. 21. January, 1999. PCR products are spotted on the array and allowed to anneal. The extension (elongation) reaction is carried out using a polymerase, a labeled dNTP and non competing ddNTPS. Incorporation of the labeled dNTP is then detected by the appropriate means. In a variation of this method suitable for use with multiplex PCR, extension is accomplished with the use of the appropriate labeled ddNTP and unlabeled ddNTPs (Pastinen et al. 1997, Genome Res. 7:606-614).

Solid phase mini sequencing has also been used to detect multiple polymorphic nucleotides from different templates in an undivided sample (Pastinen et al. 1996, Clin. Chem. 42:1391-1397). In this method, biotinylated PCR products are captured on the avidin-coated manifold support and rendered single stranded by alkaline treatment. The manifold is then placed serially in four reaction mixtures containing extension primers of varying lengths, a DNA polymerase and a labeled ddNTP, and the extension reaction allowed to proceed. The manifolds are inserted into the slots of a gel containing formamide, which releases the extended primers from the template. The extended primers are then identified by size and fluorescence on a sequencing instrument.

Fluorescence resonance energy transfer (FRET) has been used in combination with mini sequencing to detect SNPs (U.S. Pat. No. 5,945,283; Chen et al. 1997, Proc. Natl. Acad. Sci. USA 94:10756-10761). In this method, the extension primers are labeled with a fluorescent dye, for example fluorescein. The ddNTPs used in primer extension are labeled with an appropriate FRET dye. Incorporation of the ddNTPs is determined by changes in fluorescence intensities.

The above discussion of methods for the detection of SNPs is exemplary only and is not intended to be exhaustive. Further modifications of the above-mentioned methods for detection of SNPs that are within the scope and spirit of the present invention can be easily devised by the person skilled in the art, without any undue experimentation. Any method capable of detecting a single nucleotide polymorphism, including any methods as described above, can be used in present invention. In a preferred embodiment, present invention provides a method wherein said detection is accomplished by sequencing, mini sequencing, hybridization, restriction fragment analysis, oligonucleotide ligation assay or allele specific PCR.

In another embodiment present invention provides an isolated polynucleotide comprising 10 contiguous nucleotides of the ENOS gene sequence or the complement thereof, and containing at least one single nucleotide polymorphism, wherein said single nucleotide polymorphism is associated with a predisposition for accelerated ADPKD. More preferably, present invention provides an isolated polynucleotide wherein said single nucleotide polymorphism corresponds to the Glu 298 Asp polymorphism of the ENOS gene.

In accordance with the diagnostic method of the present invention, the method of present invention can be performed by using a polynucleotide capable of hybridizing to a region of the ENOS gene. The nucleotide sequence of the ENOS gene is known (see GenBank Acc. No. D 26607). Thus, a primer or oligonucleotide derived from the nucleotide sequence of the ENOS gene can be easily used for the detection of the Glu 298 Asp polymorphism.

Preferably, said polynucleotide is about 15 to 50, preferably 20 to 40, more preferably 20 to 30 nucleotides in length and comprises a nucleotide sequence of the ENOS gene, or the complement thereof, containing a single nucleotide polymorphism. Hence, in a still further embodiment, the present invention relates to a primer or probe comprising a polynucleotide as defined above.

The polynucleotide further can contain a detectable marker. Suitable markers include, but are not limited to, radioactive labels, such as radionuclides, fluorophores or fluorochromes, peptides, enzymes, antigens, antibodies, vitamins or steroids.

Specific hybridization of the above mentioned probes or primers preferably occurs at stringent hybridization conditions. The term “stringent hybridization conditions” is well known in the art; see, for example, Sambrook et al. 1989 (Molecular Cloning, A Laboratory Manual” second ed.,CSH Press, Cold Spring Harbor) and Hames and Higgins 1985 (Eds. Nucleic Acid Hybridisation, A Practical Approach”, IRL Press, Oxford.

Furthermore, in another embodiment, the present invention relates to the use of a single nucleotide polymorphism in the ENOS gene sequence, or the complement thereof, for diagnosing accelerated ADPKD in a human subject. More preferably, the invention discloses the use of a single nucleotide polymorphism corresponding to the Glu 298 Asp polymorphism of the ENOS gene.

In another embodiment, present invention relates to a diagnostic kit for carrying out the method of present invention comprising
one isolated polynucleotide of at least 10 contiguous nucleotides of the ENOS gene sequence or the complement thereof, and containing at least one single nucleotide polymorphism, wherein said single nucleotide polymorphism is associated with a predisposition for accelerated ADPKD,
suitable reagents, and
instructions for using said polynucleotide for detecting the presence of said single nucleotide polymorphism in a biological sample containing said nucleic acid.

In a more preferred embodiment, present invention describes a diagnostic kit wherein said single nucleotide polymorphism corresponds to the Glu 298 Asp polymorphism of the ENOS gene.

The kit can further comprise a means for mobilizing genomic DNA extracted from a human subject, a sample comprising a nucleotide sequence of the ENOS gene or the complement thereof, and a sample comprising a nucleotide sequence of a polymorphism of the ENOS gene or the complement thereof. These samples can both be used to control the accuracy and reliability of the detection procedure. Furthermore, the kit may contain further ingredients such as selection markers and components for selective media. For example, without limitations, the kit may contain buffer solutions, enzymes, nucleotide triphosphates and other reagents and materials necessary for the detection of polymorphisms. Additionally, the kit may contain instructions for conducting the analyses of samples for the presence of polymorphisms and for interpreting the results obtained.

The kit of the invention may advantageously be used for carrying out a method of the invention and could be, inter alia, employed in a variety of applications, e.g., in the diagnostic field or as research tool. The parts of the kit of the invention can be packaged individually in vials or in combination in containers or multi container units. Manufacture of the kit follows preferably standard procedures, which are known to the person skilled in the art.

In a further embodiment, present invention provides a method for treatment of a human subject predisposed to develop accelerated ADPKD comprising
determining the predisposition of said subject by carrying out the method of the invention, and
administrating at least one NO-enhancing compound in said subject in need of said treatment.

In another embodiment of present invention, said treatment counteracts the effect of the detected single nucleotide polymorphism. With NO-enhancing compound is meant a substrate for eNOS, a NO donor, or a mixture thereof. In a further embodiment the present invention relates to a method of treatment of a human subject predisposed to develop accelerated ADPKD wherein said NO-enhancing compound comprises an effective amount of L-arginine, a NO donor or a mixture thereof. The amino acid L-arginine is the substrate for the endothelial NOS isoform. NO donors are heterogenous, pharmacologically active substances, which release NO, a biologically active compound.

This embodiment allows abolishing or alleviating said acceleration of the disease and allows treatment of accelerated ADPKD before the onset of clinical symptoms due to the phenotype response caused by the ENOS polymorphism. The SNP found modifies a sequence in the ENOS gene, such that there is less nitric oxide (NO) produced in tissues such as endothelial cells. For example, it was shown that male subjects carrying the Glu 298 Asp ENOS polymorphism showed a decreased NOS activity in renal arteries in association with post translation modifications and a partial cleavage of eNOS. Therefore, a treatment, such as oral administration of L-arginine, the substrate for eNOS, is devised to counteract the decreased nitric oxide production due to the SNP.

With “treating predisposition to accelerated ADPKD” is meant treating a human subject afflicted with accelerated ADPKD in order to delay the progression of the disease and to postpone the age of the patient at which renal failure occurs.

For example, and without limitation, a patient with an increased risk of developing accelerated ADPKD due to the presence of a SNP in the ENOS gene could be given a treatment to increase the production of nitric oxide (NO) by, for example the oral administration of L-arginine, thus reducing the risk of developing accelerated ADPKD. L-arginine may be administered as a supplementation i.e. in the form of pills, as L-arginine-rich food or as a diet enriched with L-arginine. L-arginine supplementation, which is possible in the three aforesaid ways, is administered in a range of 6 to 9 grams per day. Preferably L-arginine is administered in such a concentration that plasma arginine reaches levels above 35 micromol/L, which are necessary to reach the Km of eNOS.

In another embodiment, the present invention comprises the administration of an effective amount of a NO donor in order to treat predisposition to accelerated ADPKD. This NO donor may be chosen from the group comprising molsidomine, S-nitroso-N-acetylpenicillamine (SNAP), S-nitrosoglutathione (GSNO) or DETA-NO.

In a more preferred embodiment, the selected NO donor is molsidomine. Molsidomine is an anti-anginic and anti-ischemic compound that is widespread applied for the prevention and treatment of angina pectoris. In addition, molsidomine inhibits blood-platelet aggregation and also decreases the blood-pressure. Molsidomine is one of the few NO donors, which can be used to treat patients with coronary and hart diseases. Its use in kidney diseases was not suggested before. It is particularly suitable as it does not induce tolerance, it does not interact with anti-hypertensive drugs, it has very few side-effects and is very well tolerated. In the context of present invention, molsidomine may be administered at the same dose that is effective on other vascular beds. Molsidomine may be administered in a daily dose of 8 to 32 mg. More preferably, molsidomine may be administered in a dose, which comprises 8 to 16 mg per day.

Molsidomine is on the market in the form of tablets containing 2, 4 or 8 mg of the active ingredient. It may be administered daily in two different doses. However, it may be advantageous from the point of view of the comfort of the patient to have galenical forms of the medicament presenting a longer therapeutic effect. This would allow reducing the number of daily intakes of the drug. Therefore, in the context of present invention, delayed-release formulations of molsidomine, which are for instance described in WO-A1-0162256, may be administered to treat predisposition to accelerated ADPKD. For example, WO-A1-0162256 discloses an oral galenical form of molsidomine with delayed-release, which contains an therapeutic quantity of molsidomine or one of its active metabolites and which shows an in vitro dissolution rate of 15 to 25% of molsidomine release after 1 hour, of 20 to 35% release after 2 hours, of 50 to 65% release after 6 hours, of 75 to 95% release after 12 hours, of more then 85% release after 18 hours and of more then 95% release after 24 hours; whereby the in vivo peak in plasmatic molsidomine comprising a a concentration of 25 to 40 ng per ml plasma, is preferably reached within 3 to 4 hours after administration. The use of these molsidomine formulations is hereby incorporated by reference.

In another embodiment, said effective amount of L-arginine, the substrate for eNOS, a NO donor or a mixture thereof is administered in a pharmaceutically acceptable composition. The pharmaceutical composition may comprise L-arginine, a NO donor or a mixture thereof and a suitable excipient for treating accelerated ADPKD in a human subject. In the case of L-arginine, the effective amount of this amino acid may be administered as a supplementation i.e. in the form of pills, as L-arginine-rich food or as a diet enriched with L-arginine. The dosing recommendations can be indicated in product labeling in order to allow the prescriber to anticipate dose adjustments depending on the considered patient, with information that avoids prescribing the wrong drug to the wrong patient at the wrong dose.

In a further embodiment, the present invention concerns the use of a NO-enhancing compound in the preparation of a medicament for treating predisposition to accelerated ADPKD in a human subject. More particularly, the present invention relates to the use of L-arginine, a NO donor or a mixture thereof in the preparation of a medicament for treating predisposition to accelerated ADPKD in a human subject.

In another embodiment, the method for treating a human subject predisposed to develop accelerated ADPKD comprises a method wherein the human subject is a male patient. ADPKD is associated with a decreased production of NO by the endothelial NO synthase (eNOS). As the production of NO through eNOS is positively regulated by estrogens (Goetz et al. 1994, Biochem. Biophys. Res. Commun. 91:5212-5216; Chambliss et al. 2000, Circ Res. 87:e44-e52), the sensitivity to NO may be gender-specific and the influence of ENOS in ADPKD may be more critical in men than in pre-menopausal women. Both endothelium-dependent vasodilation and total body production of NO decrease in men. Consequently, men may be more sensitive than women to small modifications of NO production. Therefore, the effect of the Glu 298 Asp polymorphism may be restricted to ADPKD-afflicted male patients.

The following non-limiting examples and associated drawings and tables illustrate several aspects of the present invention.

### DETAILED DESCRIPTION OF FIGURES AND TABLES

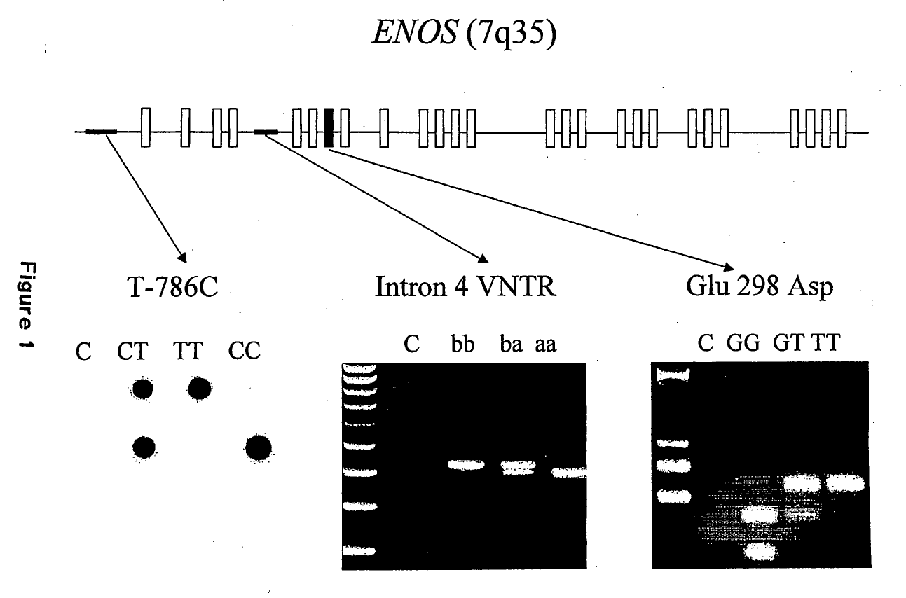

FIG. 1 shows the intron-exon structure of the ENOS gene (each box represents an exon), the localization of the 3 studied polymorphisms and representative allele-specific oligonucleotide (ASO) hybridization (T-786C) and agarose (Intron 4 VNTR and Glu 298 Asp) gels with the different genotypes corresponding to each polymorphism. The ASO were obtained after hybridization with wild type (up) or mutated (down) probes. Genotypes for the Glu 298 Asp polymorphism were obtained following digestion of the PCR products with Ban II.

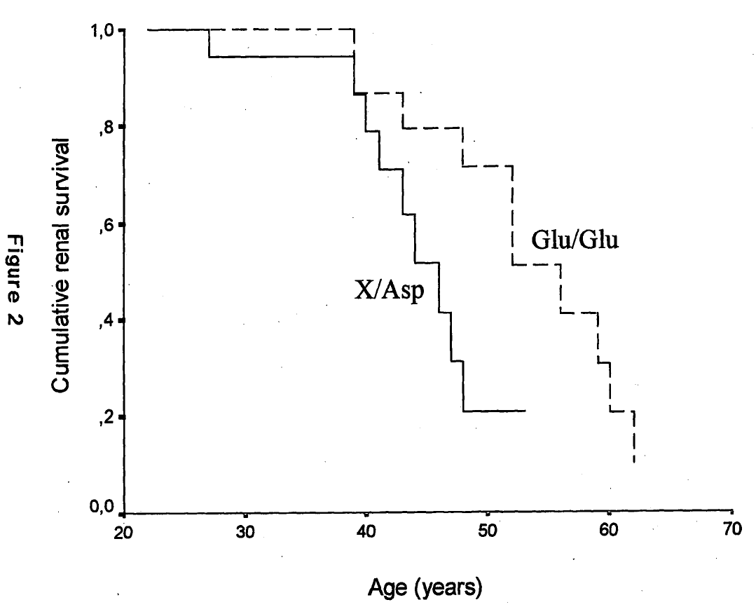

FIG. 2 represent the cumulative renal survival in the subgroup of ADPKD male patients linked to PKD1. All affected ADPKD male patients with (n=19) or without (n=19) renal failure belonging to the 27 families linked to PKD1 were included in a Kaplan-Meier analysis of cumulative renal survival, and the log-rank test was used to compare renal survival between the Glu 298 Asp genotypes.

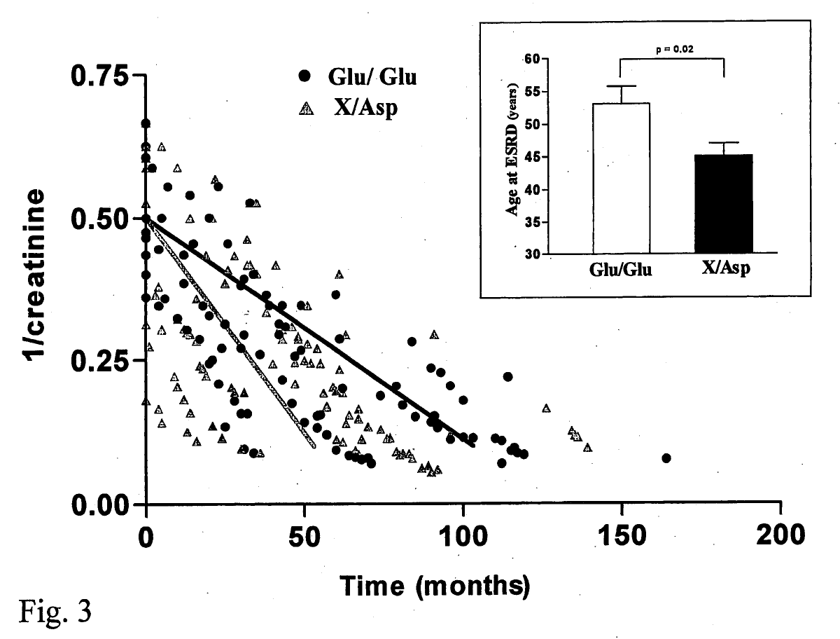

FIG. 3 shows the rate of decline in renal function, as indicated by the 1/creatinine slope, according to the Glu 298 Asp genotype in a subgroup of 22 ADPKD male patients. Each symbol represents a 1/creatinine value from an individual patient. Time represents the delay in months since the first plasma creatinine determination available for a given patient. Individual observations are symbolized by black circles for patients harbouring the Glu/Glu genotype (Glu/Glu) and grey triangles for patients harbouring the Glu/Asp or Asp/Asp genotypes (X/Asp). Individual slopes were obtained by linear regression analysis (r² was greater than 0.90 in 21/22 patients). The slopes corresponding to mean decline in renal function are indicated in black for the Glu/Glu subgroup (−0.0049) and red for the X/Asp subset (−0.0065). Inset: age at ESRD according to the Glu 298 Asp genotype in the same subgroup. X/Asp denotes Glu/Asp+Asp/Asp.

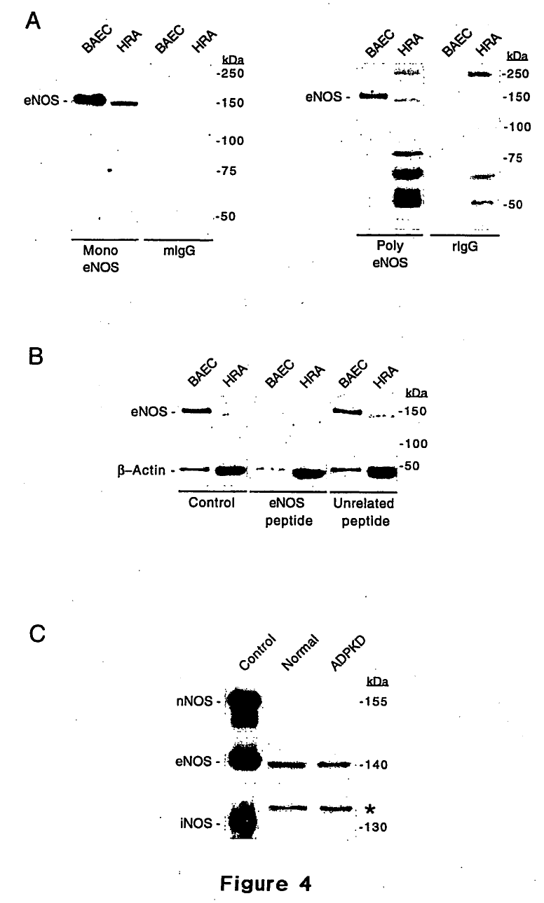

FIG. 4 represents data on the expression of endothelial NOS in renal arteries.

FIG. 4A shows immunoblot analyses of the expression of endothelial NOS (eNOS) in extracts from bovine aortic endothelial cells (BAEC, used as a positive control for eNOS) and normal human renal arteries (HRA). Two μl of BAEC lysate and 30 μg of HRA extract were run on 7.5% PAGE, transferred to nitrocellulose and probed either with monoclonal (left panel) or affinity-purified polyclonal antibodies (right panel) against human eNOS, or the corresponding mouse or rabbit non-immune IgG at the same dilution (mIgG and rIgG, respectively). The 140 kDa band corresponding to eNOS is clearly detected with both antibodies. Non-specific bands around 250 kDa and below 75 kDa are identified with non-immune IgG. The films were exposed for 5 min (antibodies) and 20 min (control IgG).

FIG. 4B depicts the, expression of eNOS in BAEC and HRA in a peptide competition analysis. Two μl of BAEC lysate and 30 μg of HRA extract were run on 7.5% PAGE and transferred to nitrocellulose. Identical strips were probed with the affinity-purified polyclonal antibodies against human eNOS in control conditions, or following preadsorption with an excess of eNOS vs. an unrelated peptide. The 140 kDa band corresponding to eNOS is competed away when primary antibodies are preadsorbed with the eNOS peptide but not when they are preadsorbed with the unrelated peptide. The signal for beta-actin was obtained after stripping the blots. The films for eNOS were exposed for 30 min.

In FIG. 4C immunoblot analyses of neuronal (nNOS), eNOS, and inducible (iNOS) NOS isoforms expression in renal arteries from normal subjects and ADPKD patients are shown. Thirty pg of extract obtained from renal arteries of a control subject and an ADPKD patient were run on 7.5% PAGE and probed with monoclonal antibodies against NOS isoforms. Positive controls include rat brain extract (nNOS), BAEC (eNOS), and macrophages (iNOS). In comparison with nNOS (155 kDa), eNOS (140 kDa) is the predominant isoform expressed in renal arteries. As previously reported (Combet et al. 2000, Kidney Int. 57:332-338), the monoclonal anti-iNOS cross-reacts with an upper band corresponding to eNOS (asterisk), but iNOS (130 kDa) is not detected in this sample.

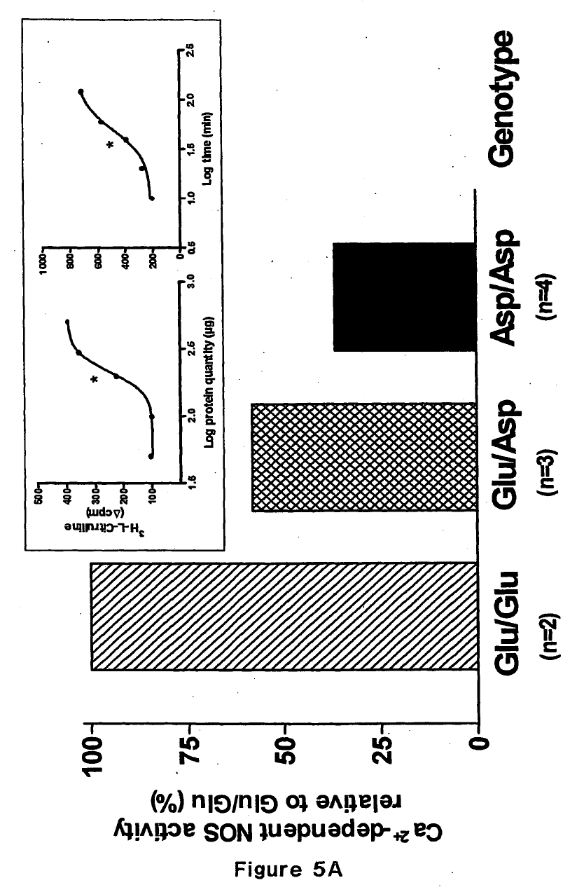

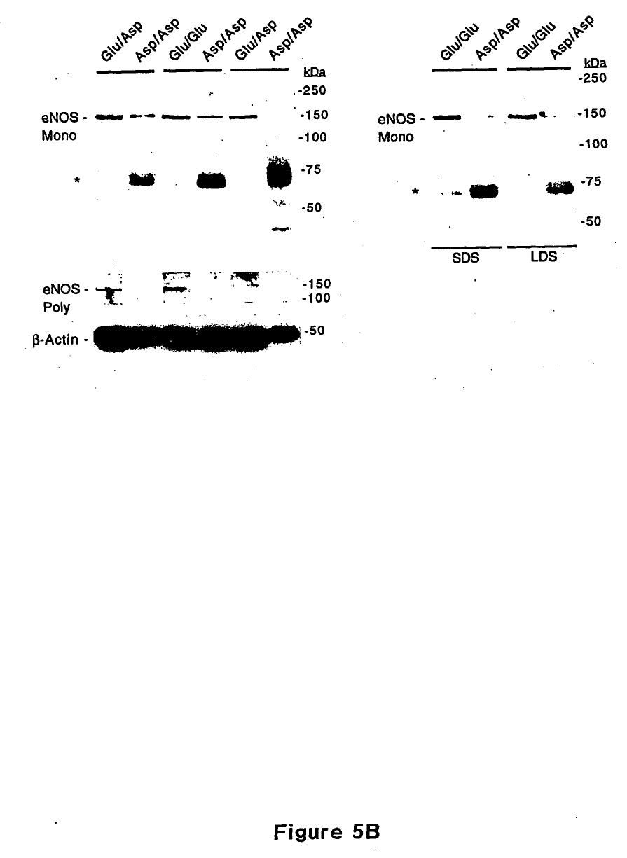

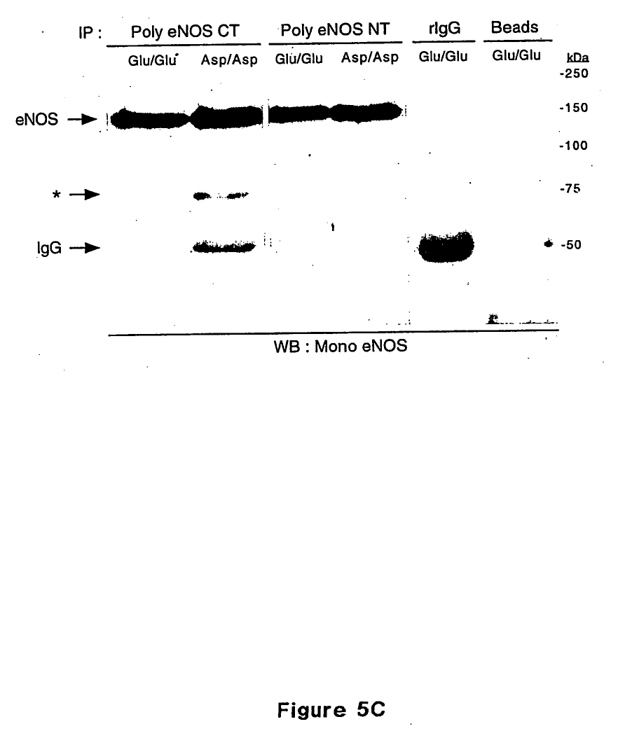

FIG. 5 represents the influence of the Glu 298 Asp polymorphism on NOS activity and expression of eNOS in renal arteries from ADPKD male patients.

FIG. 5A shows the influence of the Glu 298 Asp polymorphism on Ca²⁺-dependent NOS activity by a L-citrulline assay. The assay was performed in 9 samples, matched for age in one trio (Asp/Asp, 50 years-old vs. Glu/Asp, 49 years-old vs. Glu/Glu, 51 years-old), and three pairs (Asp/Asp, 46 years-old vs. Glu/Asp, 46 years-old; Asp/Asp, 66 years-old vs. Glu/Asp, 49 years-old; and Asp/Asp, 53 years-old vs. Glu/Asp, 53 years-old). NOS activity was measured in duplicate for each sample, with an intra-assay variability <10%, and simultaneously for the 3 pairs and the trio. Ca²⁺-dependent NOS activity, which accounted for >80% of total NOS activity in these samples, ranged from 0.02 to 0.20 pmol citrulline /mg protein/min. It was systematically lower in patients with the Glu/Asp genotype (−42%) or the Asp/Asp genotype (−63%), than in patients with the Glu/Glu genotype (taken as 100%). The ³H-L-citrulline formation as a function of protein concentration and incubation time (inset) verify that the conditions of the assay in these samples (asterisk: incubation time, 45 minutes; protein concentration, 250 μg) are in the linear part of the curve.

FIG. 5B shows the influence of Glu 298 Asp polymorphism of ENOS on the expression of eNOS in renal arteries in representative immunoblots. Three pairs of renal artery samples used for the L-citrulline assay were submitted to 7.5% PAGE and probed with monoclonal and affinity-purified polyclonal antibodies against human eNOS (left panel). In comparison with Glu/Glu and Glu/Asp samples, a significant decrease in the signal for eNOS at 140 kDa was detected in the Asp/Asp samples. Incubation with monoclonal antibodies (eNOS Mono) also showed that Asp/Asp samples were characterized by a significant increase in the amount of an immunoreactive band of lower (−70 kDa) molecular mass (asterisk). The signal for beta-actin was obtained after stripping the blot incubated with monoclonal anti-eNOS. The immunoblot pattern for eNOS in renal arteries from ADPKD male patients harbouring the Asp/Asp genotype was exactly similar when using the classical SDS conditions or the lithium dodecyl sulfate (LDS) sample buffer systems (Fairchild et al. 2001, J. Biol. Chem. 276.26674-26679) to limit acid hydrolysis (right panel). Thirty μg of samples were loaded in each lane.

FIG. 5C represents the influence of the Glu 298 Asp polymorphism on the cleavage of eNOS in renal arteries from ADPKD patients. Detergent-solubilized extracts from renal arteries were subjected to immunoprecipitation using polyclonal antibodies directed against the C-terminus (Poly eNOS CT) or N-terminus (Poly eNOS NT) of human eNOS. eNOS (140 kDa) and co-immunoprecipitated proteins (asterisk) were then probed by immunoblotting with the monoclonal antibody against human eNOS (C-terminus). Whereas full-length eNOS is detected in all immunoprecipitates, a 70 kDa protein immunoreactive for the C-terminus of eNOS is identified in the Asp/Asp sample only. Control experiments performed with non-immune rabbit IgG (rIgG) or beads only failed to immunoprecipitate eNOS.

Table 1 shows the age at ESRD and distribution of the genotypes of ENOS polymorphisms in male and female ADPKD subsets.

Table 2 depicts the allele frequencies and pairwise linkage disequilibrium between ENOS polymorphisms.

Table 3 represents the age at ESRD according to genotypes of ENOS polymorphisms in male and female ADPKD subsets (simple regression analysis).

Table 4 illustrates the age at ESRD according to combinations of T-786C and Glu 298 Asp genotypes in ADPKD male patients (multiple regression analysis).

Example 1 and 2 relate to the distribution of different polymorphisms of the ENOS gene in a population.

Example 3 provides evidence that the Glu 298 Asp polymorphism of the ENOS gene is related to ADPKD, and influences the progression of the disease and the age at ESRD.

Examples 4 and 5 show that the Glu 298 Asp polymorphism is associated with a faster renal decline in ADPKD male patients.

Example 6 provides evidence that a decrease in NOS activity in association with post-translational modification and a partial cleavage of eNOS form the molecular mechanisms underlying the influence of the Glu 298 Asp polymorphism.

### EXAMPLE 1

### Characteristics of the ADPKD Population Studied and Distribution of ENOS Polymorphisms in the Population

Patients at ESRD were recruited from September 1998 to September 2000 in Saint-Luc Academic Hospital, Brussels (Belgium); U.Z. Gasthuisberg, Leuven (Belgium); and Necker Hospital, Paris (France). All Caucasian patients affected with ADPKD on renal replacement therapy, i.e. dialysis or renal transplantation, in the 3 centers during the recruitment period were included. The diagnosis of ADPKD was established on the basis of bilateral enlarged cystic kidneys and a family history suggestive of autosomal dominant inheritance. The age at ESRD was defined as the age at starting renal replacement therapy (creatinine clearance 10 ml/min). All patients recruited in this first phase were unrelated. A detailed follow-up for at last 2 years before ESRD was available for all patients included. A total of 182 unrelated patients at ESRD were recruited. From 57 potentially informative families, linkage to PKD1 and PKD2 was established in 27 and 9 families, respectively. Patients belonging to PKD2 families were excluded from the study population. The population therefore consisted of 173 unrelated patients: 93 males and 80 females.

Method

The age at ESRD and the distribution of the genotypes of ENOS polymorphisms in male and female ADPKD subsets were studied. All data were analyzed using the SPSS statistical software (version 10.0, SPSS, Chicago, Ill.), and p values <0.05 are considered as significant. The ages at ESRD were normally distributed in males and females, and compared by two-tailed Student's t-test. For each polymorphism, allele frequencies were calculated from the genotype. Allele and genotype distribution in male and female subsets were compared by chi-square (χ²) test. Statistical analysis was performed by combining heterozygotes and homozygotes rare allele carriers.

Results

The mean age at ESRD was not significantly different in the 3 centers being the Saint Luc Academic Hospital in Brussels: 50.5±1.0, n=102; the U.Z. Gasthuisberg Hospital in Leuven: 55.0±1.8, n=35 and the Necker Hospital in Paris: 51.3±1.6, n=36 (p=0.1) a was not significantly different in males and females (Table 1).

The distribution of the 3 diallelic polymorphisms of ENOS is shown in FIG. 1 and Table 1. The genotype frequencies were not significantly different according to gender (Table 1) and center: p=0.3, p=0.1 and p=0.9 for the Glu 298 Asp, intron 4 VNTR, and T-786 polymorphisms, respectively.

TABLE 1
Age at ESRD and distribution of the genotypes of ENOS
polymorphisms in male and female ADPKD subsets.

| | Total | Male | Female | |
|---|---|---|---|---|
| | (n = 173) | (n = 93) | (n = 80) | p value |
| Age at ESRD | 51.6 ± 0.8 | 50.9 ± 1.0 | 52.3 ± 1.3 | 0.4* |
| (years) | | | | |
| Glu 298 Asp | | | | |
| Glu/Glu | 81 (46.8%) | 40 (43.0%) | 41 (51.2%) | |
| Glu/Asp | 77 (44.5%) | 45 (48.4%) | 32 (40.0%) | |
| Asp/Asp | 15 (8.7%) | 8 (8.6%) | 7 (8.8%) | 0.5° |
| Intron 4 VNTR | | | | |
| Bb | 123 (71.1%) | 64 (68.8%) | 59 (73.8%) | |
| Ba | 46 (26.6%) | 27 (29.0%) | 19 (23.8%) | |
| Aa | 4 (2.3%) | 2 (2.2%) | 2 (2.4%) | 0.7° |
| T-786C | | | | |
| TT | 61 (35.3%) | 27 (29.0%) | 34 (42.5%) | |
| CT | 89 (51.4%) | 53 (57.0%) | 36 (45.0%) | |
| CC | 23 (13.3%) | 13 (14.0%) | 10 (12.5%) | 0.2° |

The influence of gender was assessed using *student's t-test (age at ESRD) or °chi square test (genotype frequencies). Values are expressed as mean ± SEM.

### EXAMPLE 2

### Linkage Disequilibrium Between the Different ENOS Polymorphisms

The ‘Hardy-Weinberg equilibrium’ is a term used to describe the distribution of genotypes at a bi-allelic locus in a stable population without recent genetic admixture, drift, or selection pressure. The Hardy-Weinberg equilibrium is a mathematical formula that gives the relationship between gene frequencies and genotype frequencies in a population, provided a few assumptions are fulfilled (e.g. random mating) in the population studied. Deviation from the equilibrium distribution might suggest adverse survival characteristics of organisms with one of the alleles. It was investigated whether the observed genotype frequencies did deviate from the Hardy-Weinberg equilibrium.

Method

This Hardy-Weinberg equilibrium was tested by a χ² test. Pairwise linkage disequilibrium between the different polymorphisms was tested and the linkage coefficient D′ was obtained. D′ is the fraction of maximum linkage that could occur between 2 loci, given the allelic frequencies. The sign added in front of D′ is positive if the less frequent alleles at both loci are preferentially associated and negative if the less frequent allele at one locus is associated with the most frequent allele at the other locus (Thompson et al. 1988, Am. J. Hum. Genet. 42:113-124.).

Results

For each polymorphism, the observed genotype frequencies did not deviate from the Hardy-Weinberg equilibrium and comprised: Glu 298 Asp: χ²_{1df}=0.3, p=0.7; intron 4 VNTR: χ²_{1df}=0.01, p=1; T-786C: χ²_{1df}=0.7, p=0.3. The 3 polymorphisms were in significant linkage disequilibrium (p<0.001). The a allele of intron 4 VNTR was strongly associated with the Glu allele of the Glu 298 Asp polymorphism and the C allele of the T-786C polymorphism. In contrast, the Glu 298 Asp and T-786C polymorphisms were in only weak to moderate linkage disequilibrium (Table 2).

TABLE 2
Allele frequencies and pairwise linkage disequilibrium
between ENOS polymorphisms

| | Allele | Linkage disequilibrium coefficient (±D′) | | |
|---|---|---|---|---|
| Polymorphisms | frequencies* | Glu 298 Asp | Intron 4 VNTR | T-786 |
| Glu 298 Asp | 0.69/0.31 | — | — | — |
| Intron 4 VNTR | 0.84/0.16 | −0.99 | — | — |
| T-786C | 0.61/0.39 | 0.35 | 0.88 | — |

*For each polymorphism, the frequency of the most common allele is shown first. All linkage disequilibrium coefficients are statistically significant (p < 0.001).
D′ is the fraction of maximum linkage that could occur between 2 loci, given the observed allelic frequencies.

### EXAMPLE 3

### Influence of the ENOS Polymorphism on the Age at ESRD in ADPKD

The effect of the ENOS polymorphisms on the age at ESRD was investigated. The presence of different genotypes of ENOS polymorphisms was investigated in the population described in example 1. Simple linear regression analysis was used to assess the individual effect of each ENOS polymorphism on age at ESRD. In addition also multiple linear regression analysis was performed to investigate the joint effects of ENOS polymorphisms on the age at ESRD

Method

173 unrelated patients, 93 males and 80 females, were recruited as described in example 1. To determine the presence and the genotype of the Glu 298 Asp ENOS polymorphism in this population DNA was extracted from peripheral blood samples (Gentra, Minneapolis, Minn.). Polymerase chain reaction (PCR) for the ENOS polymorphism was carried out in a 20 μl volume with 100 ng of genomic DNA, 10 pM of each primer, 1.25 mM dNTP (Roche Diagnostics, Mannheim, Germany), 1 unit of Taq polymerase (Roche) and 2 μl of 10× buffer containing 15 mM MgCl₂ (Roche). Genotyping for the intron 4 VNTR was performed using primers and conditions described previously (Wang et al. 1996, Nat. Med. 2: 41-45; Wang et al. 1999, Nephrol. Dial. Transplant. 14:2898-2902). Genotyping for Glu 298 Asp was performed by PCR, followed by BanII (Life Technologies, Carlsbad, Calif.) digestion, and additional control using Mbol digestion, as described (Miyamoto et al. 1998, Hypertension, 32:3-8) Genotyping for T-786C was obtained by PCR followed by allele-specific oligonucleotid hybridization (ASO), as described earlier (Zanchi et al. 2000, Kidney Int. 57:405-413). Three patients found to harbor the 3 possible genotypes (TT, CT and CC) by sequencing were used as positive controls.

Simple linear regression analysis was used to assess the individual effect of each ENOS polymorphism (independent variable) on age at ESRD (dependent variable).

Multiple linear regression analysis was performed to investigate the joint effects of ENOS polymorphisms on the age at ESRD. Variables that significantly influenced the age at ESRD were selected by a forward stepwise procedure. The increase in the model r² was used to explain the proportion of the variance added by the each polymorphism, and allowed us to select the model which explains the highest proportion of the variance. To deal with the categorical nature of a polymorphism, dummy variables were generated for each polymorphism.

Results

Results of the genotype distribution in the assessed population are shown in Table 2. Simple linear regression analysis showed that male patients with ADPKD harboring the Asp allele of the Glu 298 Asp polymorphism had a significant, 4.5-year lower mean age at ESRD than Glu/Glu patients (Table 3). The age at ESRD was increased, though less significantly, in patients harboring the a allele of intron 4 VNTR, as compared to patients with the bb genotype. The T-786C polymorphism had no effect on the age at ESRD in male patients. In female ADPKD patients, none of the polymorphisms of ENOS had an effect on the age at ESRD (Table 3).

TABLE 3
Age at ESRD according to genotypes of ENOS polymorphisms in
male and female ADPKD subsets

| | | | Age at ESRD | |
|---|---|---|---|---|
| Polymorphisms | Genotypes | N | (years) | p value |
| Males (n = 93) | | | | |
| Glu 298 Asp | Glu/Glu | 40 | 53.5 ± 1.5 | |
| | Glu/Asp + Asp/Asp | 53 | 49.0 ± 1.2 | 0.02 |
| Intron 4 VNTR | bb | 64 | 49.6 ± 1.1 | |
| | ba + aa | 29 | 53.9 ± 1.9 | 0.04 |
| T-786C | TT | 27 | 49.6 ± 1.5 | |
| | CT + CC | 66 | 51.5 ± 1.2 | 0.4 |
| Females (n = 80) | | | | |
| Glu 298 Asp | Glu/Glu | 41 | 51.4 ± 1.7 | |
| | Glu/Asp + Asp/Asp | 39 | 53.2 ± 1.9 | 0.5 |
| Intron 4 VNTR | bb | 59 | 51.6 ± 1.4 | |
| | ba + aa | 21 | 54.2 ± 3.0 | 0.6 |
| T-786C | TT | 34 | 50.4 ± 1.8 | |
| | CT + CC | 46 | 53.7 ± 1.8 | 0.2 |

Values are expressed as mean ± SEM.

The distribution of the Glu 298 Asp polymorphism was significantly different when ADPKD male patients were distinguished according to age at ESRD, with 50 years of age being the cut-off. Patients harboring the (Glu/Asp+Asp/Asp) genotypes were overrepresented (67%, 31/46) in the subset of patients reaching ESRD before age 50, compared to patients at ESRD after age 50. (47%, 22/47).

The multiple regression analysis confirmed the effect of the Glu 298 Asp polymorphism on the age at ESRD in the male subset in models including either intron 4 VNTR (p=0.025, r²=0.08) or T-786C (p=0.006, r²=0.13) polymorphisms. The model including T-786C and Glu 298 Asp polymorphisms explains 13% of the variance of age at ESRD (r²=0.13) and the effect of the Glu/Asp+Asp/Asp vs. Glu/Glu genotypes across the 3 genotypes of T-786C is significant (p=0.006). The model including Glu 298 Asp and T-786C, the two polymorphisms in weak to moderate linkage desequilibrium, explained a higher proportion of the variance (r²) and was thus retained as the best fit model. It yielded a p value of 0.006 for combined (Glu/Asp+Asp/Asp) vs. Glu/Glu genotypes across the 3 genotypes of T-786C (Table 4).

TABLE 4
Age at ESRD according to combinations of T-786C and Glu 298
Asp genotypes in ADPKD male patients

| | | | Age at ESRD |
|---|---|---|---|
| T-786C | Glu 298 Asp | n | (years) |
| TT | Glu/Glu | 19 | 50.4 ± 1.9 |
| TT | Glu/Asp + Asp/Asp | 8 | 47.9 ± 2.5 |
| CT | Glu/Glu | 16 | 54.2 ± 2.3 |
| CT | Glu/Asp + Asp/Asp | 37 | 48.6 ± 1.5 |
| CC | Glu/Glu | 5 | 63.0 ± 5.1 |
| CC | Glu/Asp + Asp/Asp | 8 | 52.1 ± 2.2 |

Values are expressed as mean ± SEM.

The effect of the Glu 298 Asp polymorphism on the age at ESRD was mainly due to Glu/Asp patients (48.5±1.3 years, n=45). The mean age at ESRD of the 8 patients harboring the Asp 298 allele at the homozygous state was 52.1±2.9 years. Two of them reached ESRD at age 61 and 67: since they belong to non-informative families, it was impossible to rule out PKD2 linkage in these 2 particular patients. The mean age at ESRD in the remaining 6 Asp/Asp patients was 48.2±1.7 years, similar to that found in the Glu/Asp subgroup.

The effect of the Glu 298 Asp polymorphism on the age at ESRD was also investigated in the subset of male patients belonging to PKD1-linked families. In this subset (n=17), the Glu/Asp and Asp/Asp genotypes were associated with a 8-year lower age at ESRD (45.8±2.8 vs. 54.1±3.1 years, p=0.067).

Furthermore, the effect of the Glu 298 Asp polymorphism was studied in the subset of male patients linked to PKD1 and those with ESRD before the age of 45 years. In this subset (n=37), the Glu/Asp and Asp/Asp genotypes were associated with a 6-year lower age at ESRD (42.4±1.3 vs. 48.5±2.6 years, p=0.03). Like in the whole population, the effect of the Glu 298 Asp polymorphism was not observed in the subsets of female patients linked to PKD1. The distribution of the Glu 298 Asp genotypes was not significantly different in the whole ADPKD population studied and in the subgroups including PKD1-linked patients.

Conclusion

The Glu 298 Asp polymorphism is associated with a significantly lower age at ESRD in ADPKD males and is over-represented in a subset of ADPKD males reaching ESRD before age 50.

### EXAMPLE 4

### Cumulative Renal Survival Analysis in the Subgroup of ADPKD Male Patients Linked to PKD1

In addition to the analyses primarily performed on unrelated ADPKD patients at ESRD, 38 affected male patients with (n=19) or without renal failure (n=19) belonging to the 27 PKD1-linked ADPKD families were included in a Kaplan-Meier cumulative renal survival analysis. The Kaplan Meier method is a statistical procedure to analyze survival curves in subgroups of patients. It is particularly appropriate in studies involving a small number of patients.

Method

The Kaplan-Meier method was applied. The log-rank test was used to compare renal survival according to the genotype at the Glu 298 Asp locus.

Results

The analysis confirmed that cumulative renal survival (FIG. 2) was significantly lower in patients harboring the Glu/Asp+Asp/Asp genotypes (44.8±1.7, 95% Cl [41.5; 48.1], n=19) compared to patients harboring the Glu/Glu genotype (53.3±2.3, 95% Cl [48.8; 57.7], n=19, p=0.03) of the Glu 298 Asp polymorphism.

Conclusion

The Kaplan-Meier analysis performed on all affected males belonging to the PKD1-linked families shows that cumulative renal survival is lower in patients harboring the Asp allele of the Glu 298 Asp polymorphism.

### EXAMPLE 5

### Correlations Between Glu 298 Asp Genotype and Clinical Parameters of ADPKD

Clinical parameters related to renal function were obtained in subsets of ADPKD patients, in an attempt to look for clinical factors underlying the lower age at ESRD observed in patients harboring the Glu 298 Asp polymorphism.

Method

The rate of decline in renal function loss over time was indicated by the 1/plasma creatinine slope for individual patients. Patients were included in the analysis only if >5 determinations of plasma creatinine were available before ESRD, and 1/plasma creatinine slope was obtained by linear regression.

Results The profile of renal function loss was obtained in 22 unrelated ADPKD male patients (FIG. 3). As in the whole male ADPKD subset, the mean age at ESRD was significantly lower in the (Glu/Asp+Asp/Asp) subgroup (45.1±1.9 years, n=12) than in the Glu/Glu subgroup (53.1±2.7 years, n=10; p=0.02). The 1/creatinine slope was steeper in the former subgroup (−0.0065 vs. −0.0049 in the Glu/Glu subgroup).

Conclusion

The Glu 298 Asp polymorphism is associated with a faster renal decline in ADPKD male patients.

### EXAMPLE 6

### NOS Enzymatic Activities and Expression of eNOS in Renal Arteries

To substantiate the molecular mechanism underlying the influence of the Glu 298 Asp polymorphism, the expression of eNOS at the protein level was assessed in renal arteries from ADPKD patients differing for the Glu 298 Asp genotype. Because the expression of eNOS is influenced by estrogens (Majmudar et al. 2000, J. Clin. Endocrinol. Metab. 85:1577-1583) and age (Barton et al. 1997, Hypertension 30:817-824), investigations were conducted on samples matched for gender and age.

Method

Renal artery samples were taken from nephrectomy specimens in 12 ADPKD male patients at the time of transplantation, and in 4 controls (2 males, 2 females) at the time of surgery for renal cell carcinoma. Most of the sample was used for protein extraction (for NOS assay and immunoblot), as previously described (Combet et al. 2000, Kidney Int. 57:332-338). Lysates from bovine aortic endothelial cells (BAEC) were obtained from Transduction laboratories (Lexington, Ky.). The protein concentrations were determined using the Bradford method (Bio-Rad, Melville, N.Y.) with BSA as standard.

NOS isoforms were detected with mouse monoclonal antibodies against human eNOS (C-terminus) and nNOS, and mouse iNOS (Transduction laboratories) and affinity-purified rabbit polyclonal antibodies against the N-terminus (Santa Cruz Biotechnology, Santa Cruz, Calif.) or C-terminus (Transduction Laboratories) of human eNOS (Combet et al. 2000, Kidney Int. 57:332-338). Both the monoclonal (C-terminus) and polyclonal (N-terminus) antibodies were used for detecting eNOS by immunoblotting. Additional antibodies included monoclonal anti-beta-actin (Sigma, St. Louis, Mo.) and rabbit polyclonal anti-human caveolin 1 (Transduction Laboratories). Peroxidase-labeled IgG were from Dako.

NOS activity was assayed in renal artery samples by the conversion of L-[³H]-arginine (Amersham, Little Chalfont, UK) to L-[³H]-citrulline as previously described (Combet et al. 2000, Kidney Int. 57:332-338). The NOS activity (pmol citrulline produced/mg protein/min) was calculated from the counts obtained with and without 1 mM L-NMMA. Assays were performed with 1 mM CaCl₂ and, alternatively, without Ca²⁺ (0 mM CaCl₂, 2 mM EGTA, 2 mM EDTA) to measure total vs. Ca²⁺-independent NOS activities and calculate Ca²⁺-dependent NOS activity. Both inter-assay and intra-assay variability were <10%. All determinations were performed in duplicate in the samples obtained from 9 ADPKD patients.

Sodium dodecyl sulfate (SDS)-polyacrylamide gel electrophoresis (PAGE) and immunoblotting were performed using either the Laemmli loading buffer or the LDS sample buffer. Cell lysates and renal artery samples prepared as described above were separated by SDS-PAGE and transferred to nitrocellulose. The membranes were blocked for 30 min at room temperature, and incubated overnight at 4° C. with the primary antibody. After washing, membranes were incubated for 1 h at room temperature with peroxidase-labeled secondary antibodies (1:5,000 dilution), and immunoblots were visualized with enhanced chemiluminescence (Amersham). The specificity of the bands corresponding to eNOS was verified by incubation (i) with non-immune rabbit or mouse IgG (Vector); (ii) with affinity-purified anti-eNOS antibodies preadsorbed with a 5-fold excess of the cognate vs. an unrelated peptide in 100 μl of PBS for 2 h at room temperature. All immunoblots were performed at least in duplicate.

Samples from ADPKD renal arteries were homogenized in ice-cold lysis buffer (20 mM Tris-HCl, pH 7.4, 2.5 mM EDTA, 0.5% Triton X-100, 1% sodium deoxycholate, 100 mM NaCl, 10 mM NaF, 1 mM Na₃VO₄, 20 mM molybdate) containing Complete^{R} protease inhibitors (Roche Diagnostics, Brussels, Belgium), followed by a brief sonication. Insoluble material was removed by centrifugation (12,000×g, 10 min) at 4° C. Equal amounts (1 mg total protein) were incubated either with polyclonal anti-eNOS, anti-caveolin1 antibodies, or non-immune rabbit IgG (Vector) for 2 h at 4° C. After centrifugation (12,000×g, 10 min), protein G sepharose (Zymed Laboratories, San Francisco, Calif.) was added to the supernatant (50 μl per tube) and incubated overnight at 4° C. The immune complexes were washed four times and boiled in SDS-PAGE sample buffer for 5 min before immublotting as described above.

Total RNA was extracted from 5 renal arteries from ADPKD male patients matched for age (Glu/Glu, n=2; Glu/Asp, n=2; Asp/Asp, n=1) using TRIzol reagent (Life Technologies Inc, Rockville, Md.). cDNA was synthesized from total RNA using random hexamers and Superscript First-strand reverse transcriptase (Invitrogen, Groningen, The Netherlands). Sequences of matching primer pairs and TaqMan probes were selected using the Primer Express software (PE Applied Biosystems, Foster City, Calif.); probes were labeled with a reporter dye (FAM) at their 5′-ends and a quencher dye (TAMRA) at their 3′-ends. The gene specific primers were as follows: ENOS SENS: 5′-cgcagcgccgtgaag-3′; ENOS ANTISENS: 5′-accacgtcatactcatccatacac-3′; ENOS TaqMan probe: 5′-FAM-cctcgctcatgggcacggtg-TAMRA-3′; GAPDH SENS: 5′-cctgttcgacagtcagccg-3′; GAPDH ANTISENS: 5′-cgaccaaatccgttgactcc-3′; GAPDH TaqMan probe: 5′-FAM-agccacatcgctcagacaccatgg-TAMRA-3′. Real-time quantitative PCR analyses were performed in duplicate with 250 nM TaqMan probe and 300 nM of both sense and antisense primers in a final volume of 50 μl using the TaqMan Universal PCR Master Mix (Applied Biosystems). PCR conditions were 50° C. for 2 min, 95° C. for 10 min followed by 40 cycles of 15 s at 95° C. and 1 min at 60° C. Fluorescence data were obtained and analysed with the Abi PRISM 5700 Sequence Detection System instrument (PE Applied Biosystems). To exclude amplification from contaminating genomic DNA, samples of RNA that had not been reverse-transcribed were run in parallel PCR reactions: these controls always remained negative. Results were expressed as Ct (number of cycles needed to generate a fluorescent signal above a predefined threshold). Relative quantitation for a given gene, expressed as fold-variation over control, was calculated using the 2^{-ΔΔCt} formula after normalization to GAPDH (ΔCt) and determination of the difference in Ct (ΔΔCt) between control and polymorphism-bearing arteries.

Results

A panel of well-characterized monoclonal and polyclonal antibodies raised against human eNOS was used to demonstrate that eNOS is the predominant NOS isoform expressed in control and ADPKD renal arteries (FIGS. 4A-C).

The L-citrulline assay was used to determine Ca²⁺-dependent NOS activity, which relies on eNOS and nNOS, in renal artery extracts from 9 ADPKD patients matched for age (FIG. 5A). This analysis showed that Ca²⁺-dependent NOS activity was systematically decreased in renal arteries of ADPKD patients harbouring the Glu/Asp genotype (−42%, n=3) or the Asp/Asp genotype (−63%, n=4) as compared with two ADPKD patients harbouring the Glu/Glu genotype. Similar results were observed for total NOS activity, reflecting the minimal Ca²⁺-independent NOS activity in these samples. Ca²⁺-dependent NOS activity decreased with age, irrespective of the genotype.

The renal artery samples that were used for the L-Citrulline assay were submitted to SDS-PAGE to investigate the expression of eNOS by immunoblotting, using both monoclonal and polyclonal antibodies against human eNOS. As shown in FIG. 5B, the Asp/Asp samples were characterized by a significant decrease in the expression of full-length eNOS at 140 kDa. This was paralleled by a major increase in the amount of an immunoreactive band for eNOS at ˜70 kDa. The apparition of a similar, 70 kDa band in endothelial cell lysates submitted to thawing/freezing cycles suggested that it might correspond to proteolytic cleavage of eNOS. It must be noted that the immunoblot pattern for eNOS in Asp/Asp samples, including the major immunoreactive band for eNOS at ˜70 kDa, was identical when using the lithium dodecyl sulfate (LDS) sample buffer system (Fairchild et al. 2001, J. Biol. Chem. 276:26674-26679) designed to limit acidic hydrolysis (FIG. 5B).

In contrast with the modified expression of eNOS at the protein level (FIG. 5B), real-time quantitative RT-PCR revealed that ENOS mRNA expression levels were not significantly different in age-matched renal arteries belonging to the different Glu 298 Asp genotypes (1.08- and 1.12-fold the Glu/Glu artery level in Glu/Asp and Asp/Asp arteries, respectively).

The hypothesis of increased proteolytic cleavage of eNOS in renal artery samples harboring the Asp/Asp genotype was substantiated by immunoprecipitation with polyclonal antibodies directed against the N-terminus or C-terminus domains of human eNOS, followed by immunoblotting with the monoclonal antibody against the C-terminus of eNOS (FIG. 5C). Immunoprecipitation with both antibodies yielded the full-length eNOS (140 kDa) in all samples, whereas a 70 kDa fragment containing the C-terminus of eNOS was identified in the Asp/Asp sample only. Immunoprecipitation with anti-caveolin antibodies yielded full-length eNOS in both samples, whereas no signal was observed when the precipitation was performed with non-immune rabbit IgG or in absence of primary antibody (beads only) (FIG. 5C).

Conclusion

NOS activity is decreased in renal arteries of ADPKD patients harboring the Asp allele of the Glu 298 Asp polymorphism of ENOS. This decrease in NOS activity is related to a modified expression of ENOS at the protein level and to a partial cleavage of eNOS.

In conclusion, the above-described examples provide evidence that the Glu 298 Asp polymorphism of ENOS is associated with a 5-year lower mean age at ESRD. The examples show that NOS activity is significantly decreased in renal artery samples from ADPKD males harboring the Asp 298 allele, in association with post-translational modifications and a partial cleavage of eNOS. The resulting decrease in NO production can enhance the endothelial dysfunction associated with ADPKD, leading to alteration of intra-renal and/or systemic haemodynamics. This in turn will result in a faster decline in renal function.

1. A method for diagnosing a predisposition for accelerated autosomal dominant polycystic kidney disease in a human subject comprising the steps of obtaining a biological sample containing nucleic acid from said subject, and detecting in said nucleic acid the presence of a single nucleotide polymorphism in the ENOS gene sequence, or the complement thereof.

2. The method according to claim 1 wherein said nucleic acid is DNA, cDNA, RNA or mRNA.

3. The method according to claim 1, wherein said single nucleotide polymorphism corresponds to the Glu 298 Asp polymorphism of the ENOS gene.

4. The method according to claim 1, wherein said detection is accomplished by sequencing, mini sequencing, hybridization, restriction fragment analysis, oligonucleotide ligation assay or allele specific PCR.

5. An isolated polynucleotide comprising 10 contiguous nucleotides of the ENOS gene sequence or the complement thereof, and containing at least one single nucleotide polymorphism, wherein said single nucleotide polymorphism is associated with a predisposition for accelerated autosomal dominant polycystic kidney disease.

6. The isolated polynucleotide according to claim 5 wherein said single nucleotide polymorphism corresponds to the Glu 298 Asp polymorphism of the ENOS gene.

7. A method of using a single nucleotide polymorphism of the ENOS gene sequence, or the complement thereof, for diagnosing accelerated autosomal dominant polycystic kidney disease in a human subject.

8. The method according to claim 7, wherein said single nucleotide polymorphism corresponds to the Glu 298 Asp polymorphism of the ENOS gene.

9. A diagnostic kit comprising at least one isolated polynucleotide of at least 10 contiguous nucleotides of the ENOS gene sequence or the complement thereof, containing at least one single nucleotide polymorphism, wherein said single nucleotide polymorphism is associated with a predisposition for accelerated autosomal dominant polycystic kidney disease; suitable reagents; and instructions for using said polynucleotide for detecting the presence of said single nucleotide polymorphism in a biological sample containing said nucleic acid.

10. The diagnostic kit according to claim 9 wherein said single nucleotide polymorphism corresponds to the Glu 298 Asp polymorphism of the ENOS gene.

11. A method for treatment of a human subject predisposed to develop accelerated autosomal dominant polycystic kidney disease comprising the steps of determining the predisposition of said subject by carrying out the method of claim 1, and administrating at least one NO-enhancing compound in said subject in need of said treatment.

12. The method according to claim 11, wherein said treatment counteracts the effect of said detected single nucleotide polymorphism.

13. The method according to claim 11 wherein said NO-enhancing compound comprises an effective amount of L-arginine, a NO donor or a mixture thereof.

14. The method according to claim 13 wherein said NO donor is moisidomine.

15. The method according to claim 13 wherein said effective amount of said L-arginine, NO donor or a mixture thereof is administered in a pharmaceutically acceptable formulation.

16. Pharmaceutical composition comprising L-arginine, a NO donor or a mixture thereof and a suitable excipient for treating predisposition to accelerated ADPKD in a human subject.

17. A method of using a NO-enhancing compound in the preparation of a medicament for treating predisposition to accelerated ADPKD in a human subject.

18. A method of using L-arginine in the preparation of a medicament for treating predisposition to accelerated ADPKD in a human subject.

19. A method of using a NO donor in the preparation of a medicament for treating predisposition to accelerated ADPKD in a human subject.
